(19) United States
(12) Patent Application Publication
(10) Pub. No.: US 2007/0010498 A1
(43) Pub. Date: Jan. 11, 2007

(54) Method for reducing blood loss associated with cardio-pulmonary bypass surgery

(76) Inventors: Klaus Theobald, Paoli, PA (US); Brad Zerler, Yardley, PA (US); Gary Nieman, Manlius, NY (US)

Correspondence Address: HOFFMANN & BARON, LLP, 6900 JERICHO TURNPIKE, SYOSSET, NY, 11791, US

(21) Appl. No.: 11/176,571

(22) Filed: Jul. 7, 2005

(51) Int. Cl. A61K 31/65

(52) U.S. Cl. 514/152; 514/284

(57) ABSTRACT

The invention provides a method for reducing blood loss associated with cardio-pulmonary bypass surgery in a human that is about to undergo, is undergoing, or has undergone, cardio-pulmonary bypass surgery. The method comprises administering to the human an effective amount of a non-antibacterial tetracycline formulation. The non-antibacterial tetracycline formulation is administered to the human about to undergo cardio-pulmonary bypass surgery within about one month prior to the surgery, or is administered to the human that has undergone cardio-pulmonary bypass surgery within about one month after the surgery.

### BACKGROUND OF THE INVENTION

Cardio-pulmonary bypass surgery is a surgical technique used to temporarily stop the beating heart and bypass the heart from blood flow. For example, this procedure may be used in coronary bypass surgery, a type of heart surgery used to improve the supply of blood and oxygen to the heart. The surgery may be necessary when blood flow in one or more of the coronary arteries is restricted or completely stopped. The blood flow may be partially or completely blocked, e.g. due to development of atherosclerotic plaques in the arteries.

During surgery, the cardiac surgeon reopens the affected vessel or redirects the flow of blood around one or more blocked arteries. The result is an improved blood flow to the heart. The improved blood flow can relieve chest pain (angina), and decrease one's risk for heart attack and sudden cardiac death.

Cardio-pulmonary bypass surgery is also used in other situations of surgery on the open heart such as, e.g. valve replacements, etc.

A serious complication associated with cardio-pulmonary bypass surgery is blood loss. Patients that undergo cardio-pulmonary bypass surgery can lose blood during and/or after the surgery. It is reported that about half of the people who have coronary artery bypass graft surgery will require transfusion of blood or blood products.

Thus, a need exists for reducing blood loss associated with cardio-pulmonary bypass surgery.

The compound tetracycline is a member of a class of antibiotic compounds that is referred to as the tetracyclines, tetracycline compounds, tetracycline derivatives and the like. The compound tetracycline exhibits the following general structure:

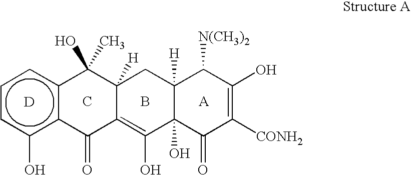

The numbering system of the tetracycline ring nucleus is as follows:

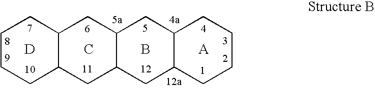

Tetracycline, as well as the terramycin and aureomycin derivatives, exist in nature, and are well known antibiotics. Natural tetracyclines may be modified without losing their antibiotic properties, although certain elements must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties.

Changes to the basic ring system or replacement of the substituents at positions 4 and 10-12, however, generally lead to synthetic tetracyclines with substantially less or effectively no antimicrobial activity. Some examples of chemically modified non-antibacterial tetracyclines (hereinafter COLs) are 4-dedimethylaminotetracyline, 4-dedimethylaminosancycline (6-demethyl-6-deoxy-4-dedimethylaminotetracycline), 4-dedimethylaminominocycline (7-dimethylamino-6-demethyl-6-deoxy-4-dedimethylaminotetracycline), and 4-dedimethylaminodoxycycline (5-hydroxy-6-deoxy-4-dedimethyaminotetracycline).

In addition to their antimicrobial properties, tetracyclines have been described as having a number of other uses. For example, tetracyclines are also known to inhibit the activity of collagen destructive enzymes produced by mammalian (including human) cells and tissues by non-antibiotic mechanisms. Such enzymes include the matrix metalloproteinases (MMPs), including collagenases (MMP-1, MMP-8 and MMP-13), gelatinases (MMP-2 and MMP-9), and others (e.g. MMP-12, MMP-14). See Golub et al., J. Periodont. Res. 20:12-23 (1985); Golub et al. Crit. Revs. Oral Biol. Med. 2:297-322 (1991); U.S. Pat. Nos. 4,666,897; 4,704,383; 4,935,411; 4,935,412. Also, tetracyclines have been known to inhibit wasting and protein degradation in mammalian skeletal muscle, U.S. Pat. No. 5,045,538, to inhibit inducible NO synthase, U.S. Pat. Nos. 6,043,231 and 5,523,297, and phospholipase A₂, U.S. Pat. Nos. 5,789,395 and 5,919,775, and to enhance IL-10 production in mammalian cells. These properties cause the tetracyclines to be useful in treating a number of diseases.

### SUMMARY OF THE INVENTION

The invention relates to a method for reducing blood loss associated with cardio-pulmonary bypass surgery in a human. The method comprises administering to the human that is about to undergo, is undergoing, or has undergone, cardio-pulmonary bypass surgery, an effective amount of a non-antibacterial tetracycline formulation, wherein the non-antibacterial tetracycline formulation is administered to the human about to undergo cardio-pulmonary bypass surgery within about one month prior to the surgery, or is administered to the human that has undergone cardio-pulmonary bypass surgery within about one month after the surgery.

### DETAILED DESCRIPTION OF THE INVENTION

Blood Loss associated with Cardio-Pulmonary Bypass Surgery

Cardio-pulmonary surgery generally involves cutting of tissue and blood vessels, which usually results in bleeding. The surgery generally is used to redirect the flow of blood from the heart and lung. The cardiopulmonary bypass surgery can, for example, be used in cardiac surgery including coronary bypass surgery, such as left or right coronary artery bypass surgery and value replacement. Blood loss associated with any cardio-pulmonary bypass surgery can be treated in accordance with the method of the present invention.

The blood loss associated with cardio-pulmonary bypass surgery refers to the loss of blood during and/or after cardio-pulmonary bypass surgery. The blood loss can, for instance, occur within one, six, twelve, twenty-four, forty-eight or more hours after the cardio-pulmonary bypass surgery. Typically, blood loss associated with cardio-pulmonary bypass surgery occurs within one month, and usually within two weeks.

In this specification, blood loss is considered to be reduced if the loss of blood is reduced by at least about 10%, preferably at least about 20%, more preferably at least about 35%, even more preferably at least about 50%, still more preferably at least about 75%, and most preferably at least about 90%. Optimally, blood loss associated with cardio-pulmonary bypass surgery is reduced such that the need for a transfusion of whole donor blood or blood products is reduced or eliminated. Examples of blood products include platelets, red blood cells, plasma, blood proteins such as albumin, and clotting factors such as factor VIII and factor IX.

Non-Antibacterial Tetracycline Formulation

In this specification, a non-antibacterial tetracycline formulation comprises a sub-antibacterial dose of an antibacterial tetracycline compound, a non-antibacterial tetracycline compound, or a pharmaceutically acceptable salt thereof.

Any antibacterial tetracycline compound may be used in the method of the present invention. Some examples of antibacterial tetracycline compounds include doxycycline, minocycline, tetracycline, oxytetracycline, chlortetracycline, demeclocycline, lymecycline. Doxycycline is preferably administered as its hyclate salt or as a hydrate, preferably monohydrate.

Non-antibacterial tetracycline compounds are structurally related to the antibacterial tetracyclines, but have had their antibacterial activity substantially or completely eliminated by chemical modification. For example, non-antibacterial tetracycline compounds have at least about two times, preferably at least about ten times, even more preferably at least about twenty five times, less antibacterial activity than that of doxycycline. In other words, non-antibacterial tetracycline compounds are incapable of achieving antibacterial activity comparable to that of doxycyline at comparable concentrations.

Any non-antibacterial tetracycline compound may be used in the method of the present invention. Some examples include those compounds disclosed generically or specifically in U.S. Pat. No. 6,638,922 issued on Oct. 28, 2003, and assigned to CollaGenex Pharmaceuticals, Inc. The tetracycline compounds disclosed in U.S. Pat. No. 6,638,922 are herein incorporated by reference.

Specific examples of non-antibacterial tetracycline compounds (COLs) include 4-de(dimethylamino)tetracycline (COL-1), tetracyclinonitrile (COL-2), 6-demethyl-6-deoxy-4-de(dimethylamino)tetracycline (COL-3), 7-chloro-4-de(dimethylamino)-tetracycline (COL-4), tetracycline pyrazole (COL-5), 4-hydroxy-4-de(dimethylamino)-tetracycline (COL-6), 4-de(dimethylamino-12α-deoxytetracycline (COL-7), 6-deoxy-5α-hydroxy-4-de(dimethylamino)tetracycline (COL-8), 4-de(dimethylamino)-12α-deoxyanhydrotetracycline (COL-9), and 4-de(dimethylamino)minocycline (COL-10).

Tetracycline compounds are either isolated from nature, or are prepared by any method known in the art. For example, natural tetracyclines may be modified without losing their antibacterial properties, although certain elements of the structure must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibacterial properties. Changes to the basic ring system or replacement of the substituents at positions 1-4 and 10-12, however, generally lead to tetracyclines with substantially less or effectively no antibacterial activity.

The term “pharmaceutically acceptable salt” refers to a salt prepared from a well-tolerated, nontoxic tetracycline compound, and an acid or base. The acids may be inorganic or organic acids of antibacterial tetracycline compounds or non-antibacterial tetracycline compounds. Examples of inorganic acids include hydrochloric, hydrobromic, nitric hydroiodic, sulfuric, and phosphoric acids. Examples of organic acids include carboxylic and sulfonic acids. The radical of the organic acids may be aliphatic or aromatic. Some examples of organic acids include formic, acetic, phenylacetic, propionic, succinic, glycolic, glucuronic, maleic, furoic, glutamic, benzoic, anthranilic, salicylic, phenylacetic, mandelic, embonic (pamoic), methanesulfonic, ethanesulfonic, panthenoic, benzenesulfonic, stearic, sulfanilic, alginic, tartaric, citric, gluconic, gulonic, arylsulfonic, and galacturonic acids. Appropriate organic bases may be selected, for example, from N,N-dibenzylethylenediamine, chloroprocaine, choline, diethanolamine, ethylenediamine, meglumine (N-methylglucamine), and procaine.

Throughout this specification, parameters are defined by maximum and minimum amounts. Each minimum amount can be combined with each maximum amount to define a range.

Dose

According to the present invention, a non-antibacterial tetracycline formulation comprising an antibacterial tetracycline compound is administered in a sub-antibacterial amount. A sub-antibacterial amount of an antibacterial tetracycline compound is any amount that results in a tetracycline plasma concentration: (i) which is effective for reducing blood loss associated with bypass surgery, but (ii) which has no, or substantially no, antibacterial activity.

A concentration of an antibacterial tetracycline compound having substantially no antibacterial activity is any concentration that does not significantly prevent the growth of bacteria. That is, a microbiologist would not consider the growth of bacteria to be inhibited from a clinical point of view.

One way in which to quantify the antibacterial activities of tetracycline compounds is by a measure called minimum inhibitory concentration (MIC), as is known by a skilled artisan.

An MIC is the minimum tetracycline concentration that inhibits the growth of a particular strain of bacteria in vitro. MIC values are determined using standard procedures. Standard procedures are, for example, based on a dilution method (broth or agar), or an equivalent, using standard concentrations of inoculum and tetracycline powder. See, for example, National Committee for Clinical Laboratory Standards. Performance Standards for Antimicrobial Susceptibility Testing—Eleventh Informational Supplement. NCCLS Document M100-S11, Vol. 21, No. 1, NCCLS, Wayne, Pa., January, 2001.

In order to inhibit the growth of a strain of bacteria in vivo, a tetracycline compound achieves a plasma concentration in excess of the MIC for the strain. Plasma concentration refers to the concentration of a tetracycline compound measured in an individual's blood sample taken at steady state. Steady state is generally achieved after dosing for five to seven terminal half lives. The half lives of different tetracycline compounds vary from hours to days.

In the methods of the present invention, an antibacterial tetracycline compound is administered in an amount that is effective, as described above, and that results in a plasma concentration which is significantly below the MIC for commonly-occurring bacteria. Such amounts are considered to have no, or substantially no, antibacterial activity. Examples of commonly-occurring bacteria that are susceptible to tetracycline are Escherichia coli (e.g., ATCC 25922 and 25922); Neisseria gonorrhoeae (e.g., ATCC 49226); Staphylococcus aureus (e.g., ATCC 29213 and 25213); and Streptococcus pneumoniae (e.g., ATCC 49619).

For example, in the present invention, an antibacterial tetracycline compound is administered in an amount that results in a plasma concentration which is less than approximately 80%, 75%, 70%, 65%, 60%, 55%, 50%, 45%, 40%, 35%, 30%, 25%, 20%, 15%, 10%, 5%, 1% or 0.5% of the MIC for the commonly-occurring bacteria mentioned above. A skilled artisan can readily determine the amount of a particular antibacterial tetracycline compound to administer to achieve such concentrations.

For example, doxycycline is administered in an amount that results in a minimum steady state plasma concentration of about 0.1 μg/ml, 0.2 μg/ml, or 0.3 μg/ml, and a maximum steady state plasma concentration of about 0.7 μg/ml, 0.8 μg/ml, or 0.9 μg/ml.

The sub-antibacterial amount of an antibacterial tetracycline compound can also be expressed by daily dose. The daily dose of an antibacterial tetracycline compound is any amount that is sufficient to produce the effective, sub-antibacterial plasma concentrations described above. Such dose can, for example, be expressed as a percentage of a minimum antibacterial daily dose.

A skilled artisan knows, or is able routinely to determine, the minimum antibacterial daily dose for antibacterial tetracycline compounds. Examples of suitable sub-antibacterial doses of antibacterial tetracycline compounds for the methods of the present invention include less than approximately: 80%, 75%, 70%, 65%, 60%, 55%, 50%, 45%, 40%, 35%, 30%, 25%, 20%, 15%, 10%, 5%, 1% and 0.5% of a minimum antibacterial dose.

Some examples of non-antibacterial daily doses of antibacterial tetracycline compounds include about 20 mg/twice a day of doxycycline; about 38 mg of minocycline one, two, three or four times a day; and about 60 mg of tetracycline one, two, three or four times a day.

There is no necessary minimum effective amount of the antibacterial tetracycline compound, as long as the amount administered is capable of reducing blood loss associated with bypass surgery. For example, when the amount is expressed as a percentage of the MIC plasma concentration, suitable minimum plasma concentrations include approximately 0.1%, 0.5%, 0.8% and 1% of the MIC plasma concentration. When the amount is expressed as a minimum actual plasma concentration, suitable actual plasma concentrations include approximately 0.01 μg/ml, 0.05 μg/ml, 0.1 μg/ml, 0.15 μg/ml, 0.2 μg/ml, 0.25 μg/ml, 0.3 μg/ml, 0.35 μg/ml, 0.4 μg/ml, 0.45 μg/ml, 0.5 μg/ml, 0.55 μg/ml, 0.6 μg/ml, 0.65 μg/ml, 0.7 μg/ml, 0.75 μg/ml, 0.8 μg/ml, 0.85 μg/ml, 0.9 μg/ml, 0.95 μg/ml, and 1.0 μg/ml. When the dose is expressed as a percentage of a minimum antibacterial daily dose, the percentage is approximately 0.1%, 0.2%, 0.5%, 1%, 1.5% and 2% of the minimum antibacterial dose.

In an embodiment, any form of doxycycline (e.g., doxycycline salts, such as doxycycline hyclate; and doxycycline hydrates, such as doxycycline monohydrate) is administered in a daily amount of, or equivalent to, from about 10 to about 60 milligrams of doxycycline, while maintaining a concentration in human plasma below the MIC.

In an especially preferred embodiment, doxycycline, a doxycycline salt, or a doxycycline hydrate is administered at a dose of, or equivalent to, 20 milligram of doxycycline twice daily. Such a formulation is sold for the treatment of periodontal disease by CollaGenex Pharmaceuticals, Inc. of Newtown, Pa. under the trademark Periostat®.

Non-antibacterial tetracycline compounds have no, or substantially no, antibacterial activity. Therefore, there is reduced indiscriminate inhibition of growth of bacteria, and the resulting threat of developing resistant bacteria. Accordingly, a non-antibacterial tetracycline formulation comprising a non-antibacterial tetracycline compound, such as the COLs discussed above, is administered at any effective dose at which side effects, if any, are acceptable.

For example, suitable maximum plasma concentrations of the COLs mentioned above include up to about 10 μg/ml, about 20 μg/ml, about 30 μg/ml, and even up to about 100 μg/ml, about 200 μg/ml and about 300 μg/ml. Suitable maximum daily doses of COLs include about 18 mg/kg/day, about 40 mg/kg/day, about 60 mg/kg/day and about 80 mg/kg/day.

A preferred COL is 6-demethyl-6-deoxy-4-de(dimethylamino)tetracycline (COL-3). COL-3 is suitably administered in doses of up to about 200 mg/day, preferably about 150 mg/day, more preferably about 100 mg/day, even more preferably about 10 mg/day, or in amounts that result in plasma concentrations of up to about 50 μg/ml, about 40 μg/ml, or about 30 μg/ml. For example, a dose of about 10 to about 20 mg/day of COL-3 produces plasma concentrations in humans of about 1.0 μg/ml.

There is no necessary minimum effective dose of COLs. Some typical minimum plasma concentrations of COLs include, for example, about 0.01 μg/ml, 0.1 μg/ml, 0.8 μg/ml, and 1.0 μg/ml. Some typical minimum daily doses of COLs include about 0.05 mg/day, about 0.1 mg/day, about 0.5 mg/day, about 1 mg/day, about 5 mg/day, or about 10 mg/day.

An advantage of the non-antibacterial tetracycline formulation useful in the method of the present invention is that they are administered at a dose which avoids side effects associated with high doses and/or long term administration of antibacterial formulations of tetracyclines. Examples of such side effects include the development of antibiotic resistant bacteria and the overgrowth of fungi and yeast. In order to avoid such side effects, antibiotics are normally administered to humans for a period of about eight to twelve days, and usually not more than about two weeks.

The non-antibacterial tetracycline formulations can more safely be administered for periods longer than antibiotic compounds. For example, the non-antibacterial tetracycline formulations can be administered for at least about three weeks, preferably at least about six weeks, more preferably at least about two months, and most preferably at least about six months. Optimally, the non-antibacterial tetracycline formulations can be administered for at least about one year.

Phototoxicity

Preferably, the tetracycline compounds have low phototoxicity, or are administered in an amount that results in a plasma level at which the phototoxicity is acceptable. The preferred amount of the tetracycline compound produces no more phototoxicity than is produced by the administration of a 40 mg total daily dose of doxycycline.

Examples of tetracycline compounds with low phototoxicity include, but are not limited to, tetracycline compounds having general formulae:

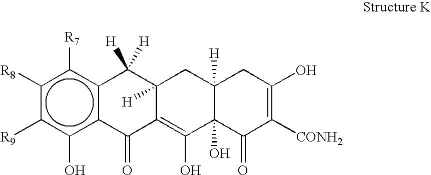

wherein: R7, R8, and R9 taken together in each case, have the following meanings:

| R7 | R8 | R9 | |
|---|---|---|---|
| hydrogen | hydrogen | amino | (COL-308) |
| hydrogen | hydrogen | palmitamide | (COL-311) |
| hydrogen | hydrogen | dimethylamino | (COL-306) |

and

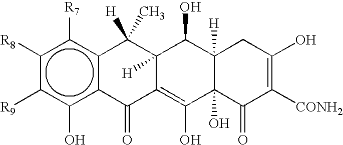

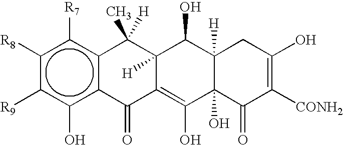

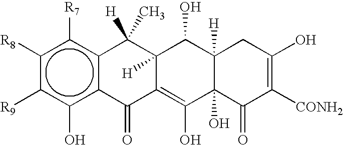

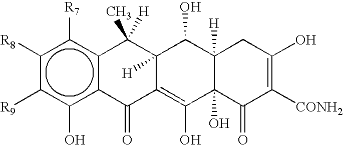

wherein: R7, R8, and R9 taken together in each case, have the following meanings:

| R7 | R8 | R9 | |
|---|---|---|---|
| hydrogen | hydrogen | acetamido | (COL-801) |
| hydrogen | hydrogen | dimethylaminoacetamido | (COL-802) |
| hydrogen | hydrogen | palmitamide | (COL-803) |
| hydrogen | hydrogen | nitro | (COL-804) |
| hydrogen | hydrogen | amino | (COL-805) |

and

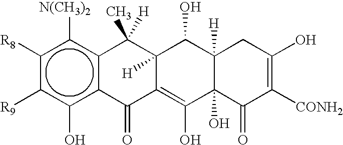

wherein: R8, and R9 taken together are, respectively, hydrogen and nitro (COL-1002).
Administration

The non-antibacterial tetracycline formulations useful in the method of the present invention is administered to a human that is about to undergo, is undergoing, or has undergone cardio-pulmonary bypass surgery.

The non-antibacterial tetracycline formulation can be administered to the human that is about to undergo cardio-pulmonary bypass surgery any time prior to the surgery. For example, administration of the non-antibacterial tetracycline formulation can occur within one month, preferably within two weeks, more preferably within one week, even more preferably within 48 hours, yet even more preferably within 24 hours prior to cardio-pulmonary bypass surgery, and most preferably within twelve hours prior to cardio-pulmonary surgery. Optimally, the human about to undergo surgery has a plasma level of a tetracycline compound that is effective in reducing blood loss.

The non-antibacterial tetracycline formulation can be administered to a human undergoing cardio-pulmonary bypass surgery. If the non-antibacterial tetracycline formulation is administered to the human during surgery, the non-antibacterial tetracycline can be administered at any time during surgery. For example, the administration of the non-antibacterial tetracycline formulation can occur within one, two, three, four, five, or more minutes after the surgery begins. Likewise, the administration of the non-antibacterial tetracycline formulation can occur within one, two, three, four, five, or more minutes before final sutures are placed.

The non-antibacterial tetracycline formulation can be administered to a human that has undergone cardio-pulmonary bypass surgery. If the non-antibacterial tetracycline formulation is administered to the human after cardio-pulmonary bypass surgery, the non-antibacterial tetracycline formulation can be administered at any time after the surgery. For example, administration of the non-antibacterial tetracycline formulation can occur within one month, preferably within two weeks, more preferably within one week, even more preferably within 48 hours, yet even more preferably within 24 hours, and most preferably within twelve hours after cardio-pulmonary bypass surgery. Optimally, the non-antibacterial tetracycline formulation is administered to the human as soon as possible after cardio-pulmonary bypass surgery (e.g., within one, two, three, four, five, ten, fifteen, thirty, sixty or more minutes).

The non-antibacterial tetracycline formulation may be administered by any method known in the art. The actual preferred amounts of a non-antibacterial tetracycline formulation in a specified case will vary according to the particular tetracycline compound used, the mode of application, the particular sites of application, and the subject being treated (e.g. age, gender, size, tolerance to drug, etc.)

The non-antibacterial tetracycline formulation may be administered systemically. For the purposes of this specification, “systemic administration” means administration to a human by a method that causes the compounds to be absorbed into the bloodstream.

Preferably, the non-antibacterial tetracycline formulation is administered orally by any method known in the art. For example, the non-antibacterial tetracycline formulation can be administered in the form of tablets, capsules, pills, troches, elixirs, suspensions, syrups, wafers, chewing gum and the like.

Additionally, the non-antibacterial tetracycline formulations can be administered enterally or parenterally, e.g., intravenously; intramuscularly; subcutaneously, as injectable solutions or suspensions; intraperitoneally; or rectally. Administration can also be intranasally, in the form of, for example, an intranasal spray; or transdermally, in the form of, for example, a patch.

For the pharmaceutical purposes described above, the non-antibacterial tetracycline formulations useful in the methods of the invention can be formulated per se in pharmaceutical preparations optionally with a suitable pharmaceutical carrier (vehicle) or excipient as understood by practitioners in the art. These preparations can be made according to conventional chemical methods.

In the case of tablets for oral use, carriers commonly used include lactose and corn starch, and lubricating agents such as magnesium stearate are commonly added. For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the non-antibacterial tetracycline formulations can be employed, and the pH of the solutions can be suitably adjusted and buffered. For intravenous use, the total concentration of the solute(s) can be controlled in order to render the preparation isotonic.

The non-antibacterial tetracycline formulation of the present invention can further comprise one or more pharmaceutically acceptable additional ingredient(s) such as alum, stabilizers, buffers, coloring agents, flavoring agents, and the like.

The non-antibacterial tetracycline formulation may be administered at intervals. For example, the tetracycline formulation may be administered one to six times a day, preferably one to four times a day, more preferably twice a day, and even more preferably once a day.

In an embodiment, the non-antibacterial tetracycline formulation containing any of the above described doses of any antibacterial tetracycline compounds or non-antibacterial tetracycline compounds, such as those mentioned above, e.g., doxycycline and COL-3, is administered by controlled release over a particular period of time, such as a 24 hour period. The level of tetracycline compound over a particular period of time is typically measured by plasma concentration, such as discussed above. Suitable controlled release formulations include delayed, sustained, and immediate (i.e., instantaneous) release.

For example, doxycycline is preferably administered in an amount of about 40 milligrams over the 24 hour period. The controlled-release 40 mg doxycycline can, for example, be formulated to contain 30 mg of doxycycline for instantaneous release and 10 mg of doxycycline for delayed release.

Methods for controlled release of drugs are well known in the art, and are described in, for example, international patent application PCT/US02/10748, which is assigned to CollaGenex Pharmaceuticals, Inc. of Newtown, Pa. and U.S. Pat. Nos. 5,567,439; 6,838,094; 6,863,902; and 6,905,708.

In one embodiment, the non-antibacterial tetracycline formulation is administered as a pharmaceutical composition comprising an active ingredient wherein the active ingredient consists essentially of a antibacterial tetracycline compound or a non-antibacterial tetracycline compound in an amount that is effective to achieve its purpose but has substantially no antibacterial activity.

What is claimed is:

1. A method for reducing blood loss associated with cardio-pulmonary bypass surgery in a human that is about to undergo, is undergoing, or has undergone, cardio-pulmonary bypass surgery, the method comprising administering to the human an effective amount of a non-antibacterial tetracycline formulation, wherein the non-antibacterial tetracycline formulation is administered to the human about to undergo cardio-pulmonary bypass surgery within about one month prior to the surgery, or is administered to the human that has undergone cardio-pulmonary bypass surgery within about one month after the surgery.

2. A method according to claim 1, wherein the non-antibacterial tetracycline formulation is administered prior to cardio-pulmonary bypass surgery.

3. A method according to claim 1, wherein the non-antibacterial tetracycline formulation is administered during cardio-pulmonary bypass surgery.

4. A method according to claim 1, wherein the non-antibacterial tetracycline formulation is administered after cardiopulmonary bypass surgery.

5. A method according to claim 1, wherein the non-antibacterial tetracycline formulation comprises an antibacterial tetracycline in a sub-antibacterial amount.

6. A method according to claim 5, wherein the antibacterial tetracycline is doxycycline.

7. A method according to claim 5, wherein the antibacterial tetracycline is minocycline.

8. A method according to claim 5, wherein the antibacterial tetracycline is tetracycline.

9. A method according to claim 1, wherein the non-antibacterial tetracycline formulation comprises a non-antibacterial tetracycline.

10. A method according to claim 9, wherein the non-antibacterial tetracycline is COL-3.

11. A method according to claim 9, wherein the non-antibacterial tetracycline is COL-308.

12. A method according to claim 1, wherein the non-tetracycline formulation reduces or eliminates need for a transfusion of blood or blood products.
